(19) United States
(12) Patent Application Publication
(10) Pub. No.: US 2019/0290117 A1
(43) Pub. Date: Sep. 26, 2019

(54) INTERFEROMETRIC FUNDUS IMAGING METHOD

(71) Applicant: Kabushiki Kaisha TOPCON, Tokyo (JP)

(72) Inventors: Zhenguo Wang, Ridgewood, NJ (US); Zhijia Yuan, Huntington, NY (US); Charles A. Reisman, Mamaroneck, NY (US); Kinpui Chan, Ridgewood, NJ (US)

(21) Appl. No.: 15/928,915

(22) Filed: Mar. 22, 2018

(51) Int. Cl. A61B 3/00; G01B 9/02; A61B 3/12; A61B 3/14

(52) U.S. Cl. CPC A61B 3/0025; G01B 9/02041; A61B 3/113; A61B 3/12; A61B 3/14; G01B 9/02004

(57) ABSTRACT

An interferometric imaging apparatus utilizes a split spectrum and/or frequency filtering process for generating fundus images. According to the split spectrum process, a bandwidth of a light source is divided into sub-spectrums of light, each used to generate pixel data for the fundus image. Data capture can thus be increased by a factor corresponding to the number of sub-spectrums. According to the frequency filtering process, a frequency filter associated with a depth of interest selectively retains data corresponding to that depth.

### BACKGROUND OF THE INVENTION

### 1. Field of the Invention

This application relates generally to fundus imaging and, more specifically, to fast generation of 2D fundus images of the eye using an interferometric imaging modality.

### 2. Description of Related Art

Fundus imaging provides essential diagnostic information in ophthalmology. From a signal detection point of view, there are three criteria for imaging performance: (1) light collection efficiency; (2) system detection sensitivity; and (3) artifacts suppression. Due to the particular structure of the eye, there are several constraints for fundus imaging. For example, both illumination and imaging aperture are limited by the pupil size, the light scattered from the retina is weak, and strong reflections from the anterior segment (particularly the cornea) can eclipse the weak signal from the retina and spoil the image contrast.

For conventional fundus imaging modalities including fundus cameras and scanning laser ophthalmoscopes (SLO), it is critical to suppress the image artifacts such as the glare from the cornea reflection, for example, by separating the illumination and imaging apertures. The pupil area is split into two parts: one for illumination, and another for imaging. However, this approach reduces the usable aperture for both illumination and imaging and, therefore, compromises the overall efficiency of the imaging performance.

Besides artifacts suppression, sufficient signal to noise ratio (SNR) may be needed for diagnostic images. Since the reflectivity of the retina is low, imaging of the retina requires either high illumination power (in the case of fundus camera) or high sensitivity with an SNR close to a shot noise limit (in the case of SLO) to generate desirable image quality. Due to safety limits, fundus cameras cannot provide high quality fundus images at video rate. In traditional SLO, special detector and data acquisition modules are employed to achieve high sensitivity, including but not limited to confocal pinhole, high gain detectors (e.g., photomultiplier tube), and avalanche diodes (APDs). However, typically such a design can only achieve high sensitivity within a certain range of the scattered light power. For a scattered signal that is not within this optimal range, some of the system performance is sacrificed, for instance, the imaging speed. Despite such compromises, shot-noise-limited high sensitivity may still not be possible considering the wide variation in retina backscattering among different people.

Recently, optical coherence tomography (OCT) has become another widely used imaging modality in ophthalmology. By utilizing a broad band light source with short coherence length, OCT can provide sub 10 μm resolution in depth. With 2-dimensional scanning, OCT can also acquire 3D volumetric images. These 3D images can further generate en-face fundus images by projection in the depth direction. However, in recent more sensitive Fourier domain OCT (FD-OCT), including spectral domain OCT (SD-OCT) based on spectrometer and swept source OCT (SS-OCT) based on tunable laser, complex ambiguities (also known as mirror image artifacts) reduce the useful depth measurement range to half in many clinical applications. This is due to the Hermitian Fourier transform of the real-valued spectral interferogram. In addition, the sensitivity may also be reduced when Fourier transforms are employed and en-face fundus images are generated based on the resulted OCT images. Due to the limited scanning speed and/or data acquisition, OCT fundus imaging has yet to achieve video rate. Still further, OCT systems are generally expensive due to the high quality and sophistication of its components such as high resolution high speed spectrometers, fast tunable lasers with large sweeping ranges, high speed digitizers, and computers for sophisticated processing. As a result, OCT fundus images are generally compromised by eye motion artifacts and insufficient for eye tracking purpose.

### BRIEF SUMMARY OF THE INVENTION

To address the limitations in existing fundus imaging modalities described above, the description herein provides an interferometric fundus imaging system and method that can approach or achieve video rate by eliminating the computation intensive Fourier transforms. The resulting image can be more sensitive than conventional OCT fundus images and useful for eye tracking applications.

In a first example, a method of imaging comprises: applying a plurality of different spectrums of light from a swept source light source to an object via a two-dimensional scanner; detecting light of each of the plurality of different spectrums of light that is backscattered by the object, detected light of each applied spectrum of light corresponding to a unique pixel of an en-face image of the object having an M×N pixel array; and generating the en-face image of the object from data corresponding to the detected light, wherein the plurality of different spectrums of light each comprise at least one unique wavelength of light.

According to various embodiments of the first example, the method further comprises: synchronizing the two-dimensional scanner with a duty cycle of the light source such that as an output of the light source changes spectrums, the two-dimensional scanner causes the light from the light source to be applied at a different location of the object; the two-dimensional scanner does not alter a location of light applied to the object while the light source is inactive; an instantaneous linewidth of the swept source light source is smaller than 0.72 nanometers; a wavelength tuning range of the swept source light source is larger than 0.017 nanometers; each pixel of the en-face image is generated by calculating the sum of the squared signal intensities for the detected light of the spectrum of light corresponding to each pixel; the en-face image is generated by normalizing pixels of the M×N pixel array corresponding to each of the at least two different spectrums; the method further comprises: frequency filtering data corresponding to the detected light to selectively retain a portion of the data corresponding to depths of interest of the object; a filtering bandwidth is adjusted based on an estimate of curvature of the object and an evaluation of the en-face image; the light is applied and detected according to an interferometric system, and the method further comprises: adjusting a path length of a reference arm of the interferometric system such that the path length of the reference arm and a path length of a detection arm of the interferometric system are equal at varying depths corresponding to a curvature of the object; the en-face image is a fundus image; the object is an eye ball; the method further comprises aligning and/or tracking an eye ball based on the generated en-face image, wherein the method is performed at least in part by an interferometric system; and/or the method further comprises: digitizing each detected spectrum at at least 15 sample points within the spectrum, the en-face image being generated at least in part from the digitized sample points.

In a second example, a method of imaging comprises: detecting spectrums of light that are backscattered by an object at various depths of the object, each detected spectrum of light corresponding to a unique pixel of an en-face image of the object having an M×N pixel array and being output by a swept source light source; filtering data corresponding to the detected spectrums of light by applying a frequency filter corresponding to a depth of interest; selectively retaining the filtered data; and generating the en-face image of the object by performing a statistical calculation on the selectively retained data.

In various embodiments of the above example, the spectrums of light are the same; the spectrums of light comprise at least two different spectrums within the bandwidth of the swept source light source, the at least two different spectrums each comprising at least one unique wavelength of light; the method further comprises: synchronizing the two-dimensional scanner with a duty cycle of the light source such that as an output of the light source changes spectrums, the two-dimensional scanner causes the light from the light source to be applied at a different location of the object; the two-dimensional scanner does not alter a location of light applied to the object while the light source is inactive; an instantaneous linewidth of the swept source light source is greater than 0.72 nanometers; a wavelength tuning range of the swept source light source is less than 0.017 nanometer; each pixel of the en-face image is generated by calculating the sum of the squared signal intensities for the detected light of the spectrum of light corresponding to each pixel; the en-face image is generated by normalizing pixels of the M×N pixel array corresponding to each of the at least two different spectrums; a bandwidth of the frequency filter is adjusted based on an estimate of curvature of the object and an evaluation of the en-face image; the light is applied and detected according to an interferometric system, and the method further comprises: adjusting a path length of a reference arm of the interferometric system such that the path length of the reference arm and a path length of a detection arm of the interferometric system are equal at varying depths corresponding to a curvature of the object; the en-face image is a fundus image; the object is an eye ball; the method further comprises aligning and/or tracking an eye ball based on the generated en-face image, wherein the method is performed at least in part by an interferometric system; the method further comprises: digitizing each detected spectrum at at least 15 sample points within the spectrum, the en-face image being generated at least in part from the digitized sample points.

These and other embodiments are described in more detail below.

### BRIEF DESCRIPTION OF SEVERAL VIEWS OF THE DRAWING

FIG. 1 illustrates a simplified interferometric setup;

FIG. 2 illustrates the relationship between a wavenumber (k) and a time (t) for a tunable laser;

FIGS. 3A and 3B illustrate the relationships between depth and frequency response;

FIGS. 4A and 4B illustrate example images generated according to the present disclosure where the reference and sample arm path lengths are the same within a retina, and above the retina;

FIG. 5 illustrates selecting a signal from a region of interest (according to depth) with frequency filters;

FIG. 6 illustrates one example of the imaging modality of the present disclosure;

FIGS. 7A and 7B illustrate a scanning scheme for 2D en-face image generation;

FIGS. 8A and 8B illustrate the relationships between scanning position and time;

FIG. 9 illustrates a split spectrum technique and sub-bands within a tuning range of a source;

FIGS. 10A and 10B illustrate an acquired signal split into 10 sub-bands and a resulting en-face image of the eye;

FIG. 11 illustrates a timing diagram for a scanning controller;

FIGS. 12A and 12B illustrate interleaving active and dummy pixels of an image;

FIG. 13 illustrates one technique for interleaving illustrated in FIGS. 12A and 12B;

FIG. 14 illustrates another technique for interleaving illustrated in FIGS. 12A and 12B; and

FIG. 15 is a flow diagram illustrating a method for imaging.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

The present disclosure herein describes a new interferometric imaging modality that enables fast generation of 2D fundus image by accumulation of a filtered interferogram, reducing or eliminating the need for sophisticated computation to generate an A-profile. One particular implementation may use a split-spectrum technique to increase the imaging rate by a factor of K, where K is the number of sub-bands of the split spectrum and is flexibly adjustable according to system specifications and imaging requirements. It should be noted that because the imaging modality described herein utilizes an interferometric setup, it may be implemented alongside other interferometric modalities, utilizing a single interferometric setup. While the disclosure contained herein illustrates the present invention with respect to ophthalmic imaging and OCT, it is to be understood that this is not a limiting embodiment. That is, interferometric techniques are also used, for example, in astronomy, spectroscopy, oceanography, and seismology.

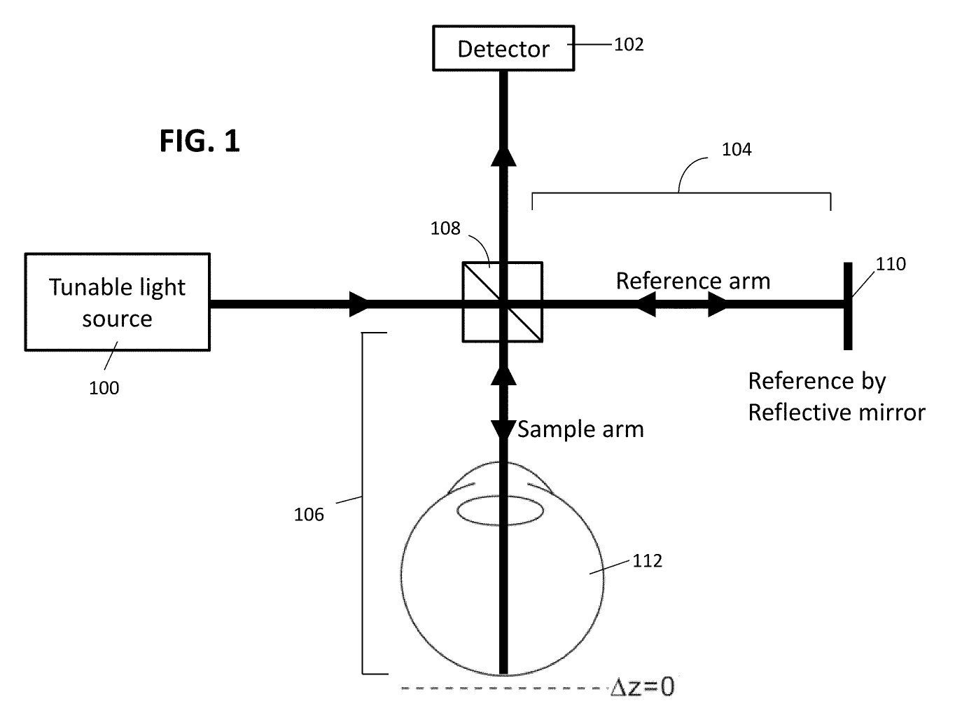

Interferometric imaging modalities, such as OCT, rely on the principle of interferometry. A simplified interferometric setup is shown in FIG. 1. An interferometer is comprised of five main elements: a source 100, a detector 102, a reference arm 104, a sample arm 106, and a beam splitter 108. In the example of FIG. 1, the source is a tunable light source 100 that outputs a bandwidth of electromagnetic waves, such as light waves. The electromagnetic waves travel through the beam splitter 108, such as a partially reflecting mirror, that splits the waves into two beams that travel through respective optics (e.g. optical fibers) constituting the reference and sample arms 104, 106. A mirror 110 at the end of the reference arm 104 and an object to be imaged 112 at the end of the sample arm 106 each reflect light back through their respective arms, then through the beam splitter 108 and into the detector 102. Depending on the lengths of the reference and sample arms 104, 106, the respective angles of incidence with the mirror 110 and the object 112, and the frequency of the waves generated by the source 100, the waves returning from the sample and reference arms 104, 106 may be in or out of phase with each other. If the waves are in phase, they will undergo constructive interference. However, when the waves are out of phase, they will undergo destructive interference. As the source 102 changes frequencies and/or the reference arm 104 length changes, the resulting interference patterns can provide meaningful information about the object to be imaged 112.

When backscattered light from a sample arm is combined with back-reflected light from a reference arm, the intensity of the detected light is determined according to equation (1):

I=Iᵣ+Iₛ+2\sqrt{IᵣIₛ} cos(Δz·k+ϕ) (1)

where Iₛ is the intensity of the backscattered light from the sample arm and Iᵣ is the intensity of the back-reflected light from the reference arm. Usually Iₛ has very low negligible power and Iᵣ can be removed as a constant background signal from the reference arm. Therefore, the interference signal, i.e., the interferogram, is determined according to equation (2):

I=2\sqrt{IᵣIₛ} cos(Δz·k+ϕ) (2)

When using a tunable light source, equation (2) can be rewritten according to equation (3):

I=A cos(Δz·R·t+ϕ′) (3)

where ϕ′=Δz·kₛ+ϕ, Δz is the difference in the respective path lengths of the sample and reference arms, and A is the interferogram amplitude A=\sqrt{IᵣIₛ}. It should be noted that, unlike frequency domain OCT applications, the system and method described herein do not require a Fourier transform or other method to convert the signal to the frequency domain and do not rely on a short coherence gate.

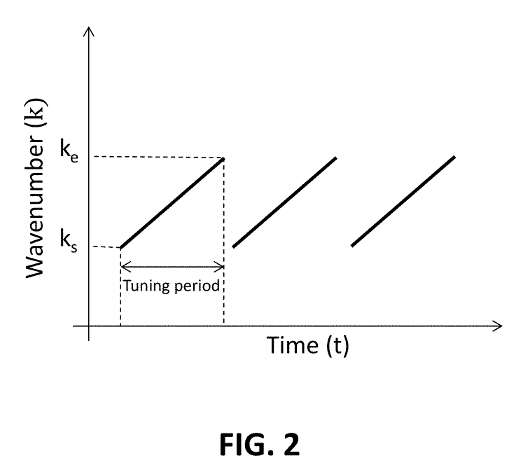

Typically a tunable laser, a laser which emits light over a particular bandwidth (its tuning range), is characterized by a wavelength λ, while a wavenumber k is widely used in interference descriptions. As wavelength and wavenumber are directly related through k=2π/λ, both k and λ are used in this disclosure and are interchangeable according to the above relationship. The relationship between the wavenumber (k) and the time (t) for a tunable laser is shown in FIG. 2. As shown on the y-axis, kₛ and kₑ are the starting and ending wavenumbers of the tunable laser, respectively, as the laser sweeps through its tuning range. To illustrate the principle, k is assumed to be linearly tuned in time. Therefore, for each tuning period, the instantaneous wavenumber is determined according to equation (4):

k=kₛ+R·t (4)

where R is the tuning speed of k,

$$\frac{dk}{dt} ,$$

and is constant in this example. It is noted that in the case of a typical tunable laser, R may not be a constant, which will then result in nonlinear tuning of k in time t. Such nonlinearity broadens the bandwidth of the interferogram, which can be readily accommodated by adjusting signal processing accordingly.

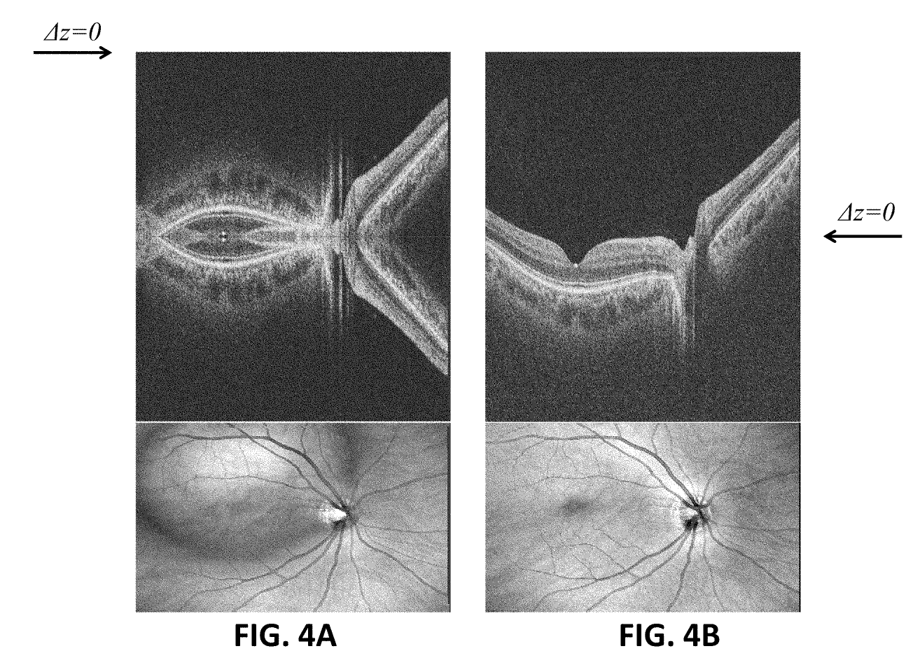

In addition, as illustrated in FIG. 1, the path length difference Δz between the sample and reference arms can be set such that the path length match point (where Δz=0) is inside of the sample. This is generally not possible for OCT due to the mirror image problem. However, such a configuration is advantageous in the system herein by lowering the requirement for detection bandwidth. As a result, sensitivity, and thereby a resulting fundus image, can be improved. FIGS. 4A and 4B illustrate OCT and corresponding fundus images according to the present disclosure that would be generated if OCT processing were performed when Δz=0 within a retina (FIG. 4A), and above the retina (FIG. 4B). It is noted that the fundus images based on the present disclosure are at least comparable under both conditions. Meanwhile, the OCT image in FIG. 4A is degraded due to mirror image and generally not useful at all.

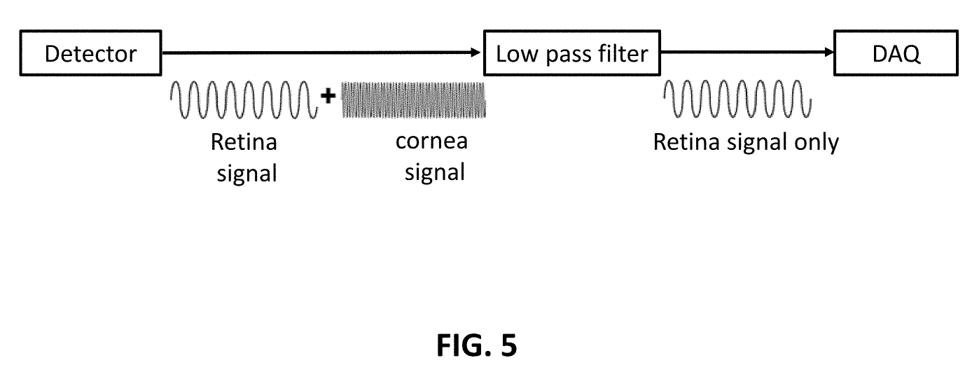

According to equation (3), the frequency of the interferogram increases as Δz, the difference in the respective path lengths of the reference and sample arms, increases. Therefore, it is possible to selectively keep the signal from a region of interest (according to depth) with standard signal processing techniques (e.g., frequency filters) and remove the others which may appear as imaging artifacts. An example of such a technique is illustrated in FIG. 5. For example, when imaging the eye, by adjusting the position of the sample and reference mirrors, the retina can be arranged at a position close to the optical path lengths matching point where Δz=0. As a result, Δz for imaging the cornea is larger than Δz for imaging the retina. Therefore, the interferogram generated by the cornea has higher frequency than the interferogram of the retina. It is then possible to suppress the interferogram from the cornea by using a low pass filter. Thereafter, the filtered interferogram can be further processed to generate a representation fundus image that is free of cornea reflection artifacts.

It is noted that the Δz corresponding to the retina and cornea can be reversed because the depth of Δz=0 is determined based on the path length of the reference arm. That is, the optical path length such that Δz=0 can be set to be the length to the cornea. In such a case, the cornea generates an interferogram with a lower frequency, which can be removed by a high pass filter. However, because a system with bandwidth at low frequency is often much easier to implement, it may be preferable to set Δz for the cornea to be larger than Δz of the retina (the length of the reference arm equals the length of the detection arm to the retina).

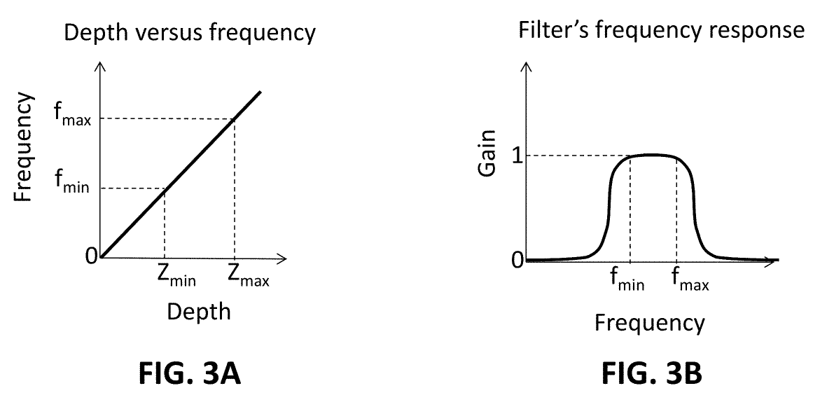

It is also noted that because of the above relationships, the filter's frequency response can be flexibly specified to select signals from specific depth. For instance, as shown in FIGS. 3A and 3B, if a desirable retina structure to be imaged ranges from Zₘᵢₙ to Zₘₐₓ in depth the signal from the retina will have a frequency band that ranges from fₘᵢₙ to fₘₐₓ. A bandpass filter thus can be customized to have cutoff frequencies of fₘᵢₙ and fₘₐₓ at low and high frequency side respectively to retain only the retina signal.

When the field of view of the fundus image increases, a dynamic configuration may be desirable to accommodate the curvature of the eye ball. According to a first embodiment, the filter bandwidth is adjusted based on a set of parameters determined by a standard model eye to estimate the curvature of the eye ball. In such an implementation, it is possible to further fine tune the filter bandwidth dynamically by utilizing a feedback loop based on evaluation of the resulting fundus image. According to a second embodiment, the path length of the reference arm is dynamically adjusted (instead of filter bandwidth) so that the signal from the region of interest (ROI) will always fall within the filter bandwidth. As such, the portion of the eye in which the path lengths are equal changes to accommodate curvature of the eye.

In the context of signal processing, and particularly time-frequency analysis, the uncertainty principle imposes the following condition on any real waveform: Δf·Δt≥1, where Δf is a measure of the bandwidth (Hz), and Δt is a measure of time duration (second). In the case of the present disclosure, the corresponding variables are Δz and Δk, respectively. Thus, Δk is the tuning range in wavenumber according to equation (5):

Δk=|2π/λs−2π/λe|≈2πΔλ/λ₀² (5)

where λₛ and λₑ are the starting and ending wavelength of the tunable laser respectively, λ₀ is the center wavelength, and Δλ=|λs−λe| is the wavelength tuning range. For fundus imaging of the eye, the retina and the cornea are about 32.4 mm (24 mm physically with refractive index of ˜1.35) apart according to the averaged axial length of human eye. Therefore, to separate retina signal from cornea reflection, Δk is determined according to: Δk≥π/Δz=π/32.4 mm⁻¹. For instance, for a near infrared light source with a center wavelength ˜1050 nm, the wavelength tuning range is found to be Δλ≥0.017 nm to separate cornea glare from retina signal. For visible light centered at 500 nm, this range is reduced to 0.004 nm. Compared to typical tunable ranges of OCT systems (on the order of tens of nanometers), the above tuning range for the present disclosure makes it possible to employ faster and less expensive tunable light sources by reducing the burden to provide large tuning range. In other words, by having a tunable range less than that for typical OCT systems, cheaper and fast light sources can be used. Further, it is possible to effectively increase imaging speed with a typical tunable laser for OCT system by splitting the spectrum of the tunable laser into small portions that can still satisfy the above ranges.

In a system with a tunable laser as the light source, the amplitude of the interference signal (and therefore the SNR) is further affected by two more factors: (1) coherence length of the tunable laser, and (2) the electrical bandwidth of the system. To avoid or minimize the retinal signal loss, the coherence length of the tunable laser should be large enough to accommodate the retina structure and its curvature. The governing formula is derived from the formula of the coherence length as shown in equation (6):

$${l}_{c} = \frac{0.44 \cdot {λ}^{2}}{δλ} > {Z}_{r} , therefore , δλ < \frac{0.44 \cdot {λ}^{2}}{{Z}_{r}} \qquad ( 6 )$$

where Zᵣ is the depth range in free space that contains the desirable retina structure, and δΔ is the instantaneous linewidth of the tunable laser. For instance, with a light source having a center wavelength ˜1050 nm, when the retina is placed close to the path length match position (Δz=0) and the Zᵣ is estimated to be ˜1.35 mm (1 mm physically with refractive index of ˜1.35), the instantaneous linewidth is found to be δΔ<0.36 nm to avoid the retina signal loss.

In the present invention, because the depth resolvable cross sectional tomography is not required for fundus image, the path length match position can essentially be set inside of the retinal structure, thereby avoiding the mirror image problem. As a result, the required instantaneous linewidth can be further relaxed to 2δΔ. In the example above, the instantaneous linewidth would be 2δλ<0.72 nm to avoid the retina signal loss. Compared to a typical corresponding instantaneous linewidth much less than 0.1 nm in OCT systems, this instantaneous linewidth for the present disclosure can be larger than prior systems. Because the instantaneous linewidth can be greater than the 0.1 nm of typical systems, it is more tolerable and thus makes it possible to employ faster and less expensive tunable light sources that are generally not useful for OCT systems.

To acquire the interferogram, the optical signal is converted to an electrical signal by a detector and/or a data acquisition device in the interferometric setup according to equation (3). The detector and data acquisition response frequency should be sufficient for resulted signal from retina in order to maintain the interferogram from the retina. In addition, the detector and data acquisition device should have frequency bandwidth according to: f_{BW}>Zᵣ·R/2π, where Zᵣ is depth range in free space that contains the desirable retina structure. When the path length match position is set inside of the retinal structure, the cutoff frequency can be further reduced by half.

After the artifacts are removed, a fundus image can be rendered with further signal processing, either in analog or digitized format. Digitization of the signal leverages the wealth of sophisticated digital signal processing techniques and therefore is further explored in detail. For instance, each pixel of the fundus image can be calculated as a statistical result, e.g., the sum of square of the signal within the sweeping range at the location(s) corresponding to the pixel. For example, according to one embodiment, at least 15 sample points within the bandwidth for each pixel on the fundus image are digitized and used to generate the fundus image. The insights gained from digitized signal processing, however, can apply equally to implementations based on analog signals.

The above description discusses the use of a tunable laser where the interferogram is frequency modulated in the time domain. However, it is noted that a broadband light source can also be used in the invention. In such a case, the interferogram is frequency modulated in the spectral domain. As a result, for broadband light source implementations, signal processing, such as noise filtering, should be done in the spectral domain instead of the time domain.

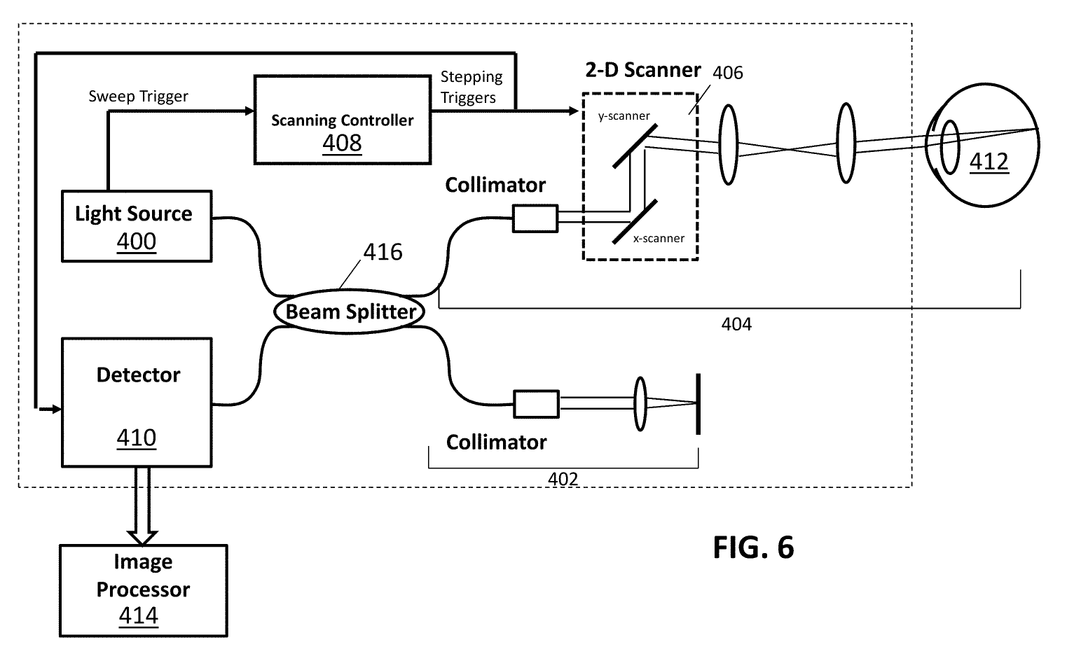

One example of the implementation of the modality of the present disclosure is shown in FIG. 6 and can include at least the following components: a swept source laser 400 as the rapid wavelength tuning laser for illumination; an interferometric setup with reference and sample arms 402, 404 formed by a beam splitter 416 to generate interferograms; a 2D scanner 406 in the sample arm for fast 2D flying spot scanning over the fundus; a scanning controller 408 that controls the 2D scanner 406 and synchronizes the scan with the laser tuning; and a detector 410 that converts light to electrical signal for processing. The detector 410 may be a detector and/or data acquisition device capable of detecting back-scattered or back-reflected light of wavelengths emitted by the swept source laser 400 or other light source used. An eye 412 to be imaged and an image processor 414 are also shown. The image processor used herein refers to any, or part of any, electrical circuit comprised of any number of electrical components, including, for example, resistors, transistors, capacitors, inductors, and the like. The circuit may be of any form, including, for example, an integrated circuit, a set of integrated circuits, a microcontroller, a microprocessor, a collection of discrete electronic components on a printed circuit board (PCB) or the like. The processor may also stand alone or be part of a computer used for operations other than processing image data. It should be noted that the above description is non-limiting, and the examples are but only a few of many possible image processors envisioned.

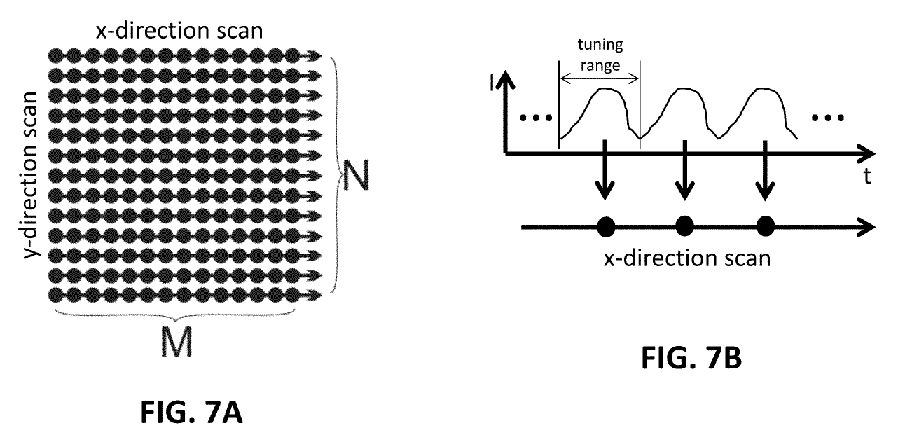

To generate the fundus image of an eye, a 2D scanning scheme is usually implemented in the sample arm 404 for fast flying spot scanning over the fundus. For example, to generate a fundus image with M×N pixels, the x-direction scan of the 2D scanner 406 will run M steps for each horizontal line, and the y-direction scan of the 2D scanner 406 will run one step forward after each x-line scan for N steps, as illustrated in FIG. 7A. As shown in FIG. 7B, the x-direction scan can be synchronized with the tunable laser source so that each pixel (step) corresponds to one spectral interferogram which is detected during one laser tuning period. This synchronization is controlled by scanning controller 408, which receives a sweep trigger signal from the light source 400 indicating the beginning of a tuning period and outputs a stepping trigger to the 2D scanner 406. Each pixel is acquired by a detector 410 from a whole spectrum and its value is calculated, by processing by an image processor 414, the acquired interferogram within the sweep range (for example, by acquiring the sum of squares of each point).

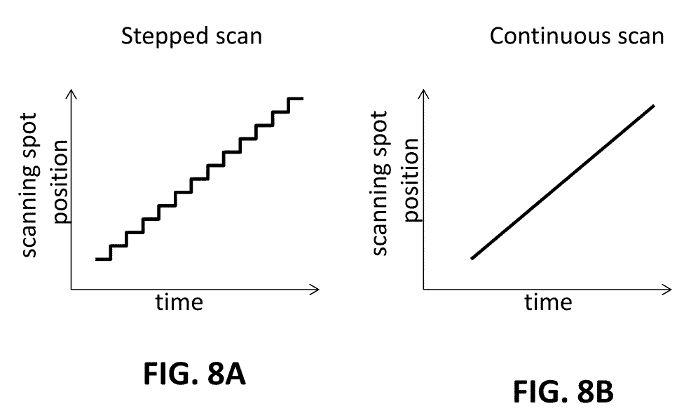

In an ideal case, the 2D scanner 406 should stop at each scanning spot until the signal of this spot is acquired, then jump to the next spot, as illustrated in FIG. 8A. However, due to the inertia of the 2D scanner 406, the actual scan is often performed continuously without stopping at each spot, as illustrated in FIG. 8B. It is noted that although continuous scanning might cause a blurring effect along the scanning direction as the acquired signal is from a segment of the scanned line instead of a fixed spot, this blurring is ignorable if the segment size is smaller than the designed image resolution. Therefore, continuous scanning is also applicable to the present disclosure.

Due to the hysteretic nature of tunable lasers, the lasing performance is different depending on the sweeping direction. Additionally, tunable lasers are typically optimized for sweeping from short to long wavelengths. As a result, at the end of the tuning period, the laser requires a return time during which laser output is usually suppressed. This inactive period, together with the linearity requirement, reduces the duty cycle of the tunable laser to be less than 100%, typically ˜50%.

As the duty cycle of a tunable laser is much lower than 100%, continuous transverse scanning may not be the optimal scanning protocol since some dummy pixels (pixels having little to no intensity/background noise only) are acquired during the inactive period of the laser. To address the problem, the scanning controller 408 can be used to control the 2D scanner 406 at the sample arm to either exclude the dummy pixels or minimize the effect of dummy pixels.

One approach to address this problem is to set up the scanning controller to generate new sweep triggers according to each sub-band (also applicable for entire tuning range without splitting), which synchronize the 2D scanner 406 with the swept laser source 400 and the data acquisition at detector 410. In doing so, the starting and ending wavenumbers/wavelengths of each sub-band are consistent during the whole scanning process and the 2D scanner 406 steps for each sub-band and stops when the laser 400 is inactive. As a result, each scanning spot corresponds to one sub-band.

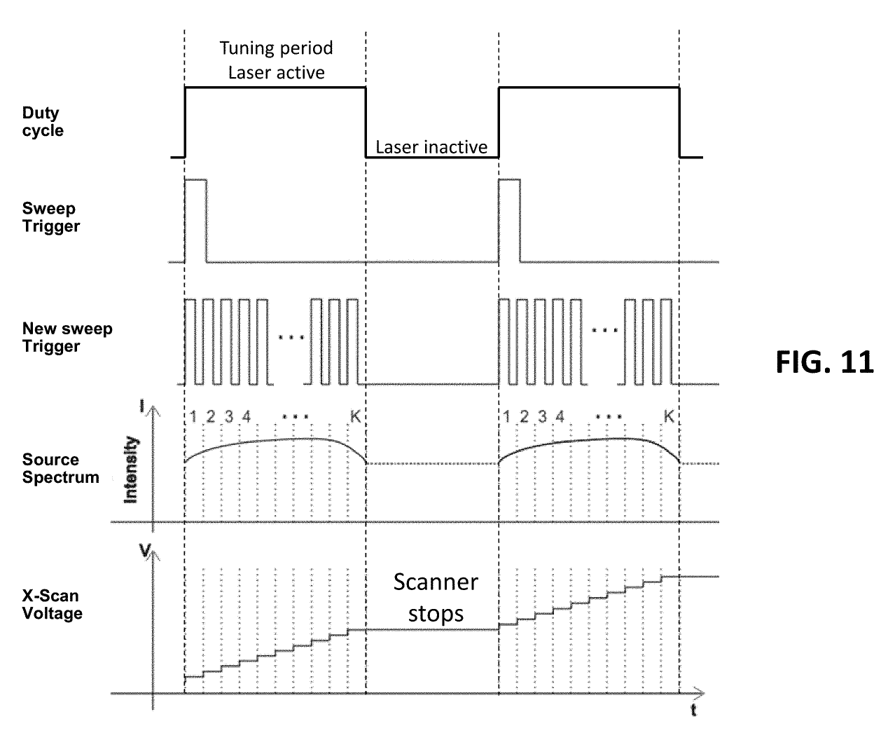

The timing diagram of such a scanning controller that addresses the problem of a limited duty cycle is shown in FIG. 11. The tuning trigger is usually generated by the swept laser source 400 to mark the start of the spectrum for each tuning period. The scanning controller 408 can generate new triggers, for example, K pulses, based on this tuning trigger. As the new trigger is also used to trigger the scanner stepping and data acquisition, the spectrum is then split into K sub-bands for each scanning spot. When the laser 400 is inactive, there is no trigger or pulse, therefore, the 2D scanner 406 stops to avoid dummy pixels in the image.

For high speed imaging, the “stop-and-start” scan control requirement previously described may be practically difficult due to the inertia and hysteresis of mechanical scanners. However, it is also possible to interleave the active scan (scan during the time when laser is active) and the dummy scan (scan during the time when laser is inactive) so that the dummy pixels (pixels acquired by dummy scan) always have active pixels (pixels acquired by active scan) next to them. As a result, each dummy pixel can be interpolated or otherwise determined based in part on its neighboring active pixels.

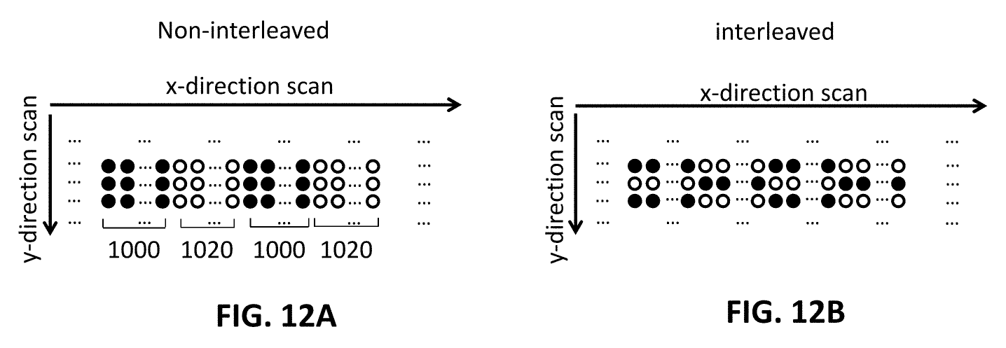

This technique is illustrated in FIGS. 12A and 12B. The filled circles 1000 are active pixels acquired when the laser 400 is active, the open circles 1020 are dummy pixels acquired when the laser 402 is inactive. Without interleaving the active and dummy pixels (FIG. 12A), the region that contains dummy pixels 402 results in blank areas in the image. By interleaving active and dummy pixels (FIG. 12B), the dummy pixels always have neighboring active pixels. As long as the image is oversampled in the y-direction, the dummy pixels can be replaced by, for example, interpolated values using their neighboring active pixels. There are many ways to generate the interleaved scanning pattern. Here two methods are described below that rely on controlling time and position, respectively.

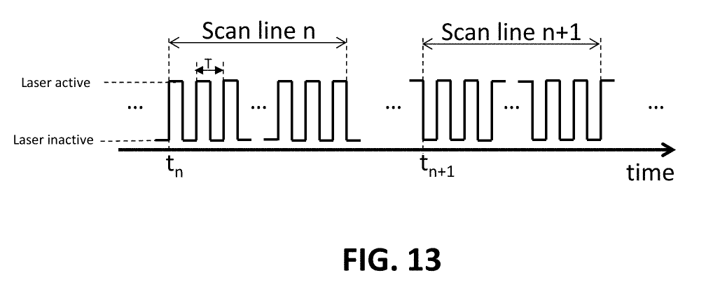

First, as shown in FIG. 13, the time delay between sequential scan lines is set according to equation (7):

$${t}_{n + 1} - {t}_{n} = m \cdot T + \frac{T}{2} , \qquad ( 7 )$$

where m is a positive integer, and T is the laser's duty cycle. As such, if the laser status at the start of scan line n is active, after

$$m + \frac{T}{2}$$

cycles when the scan line n+1 starts, the laser status becomes inactive. Or if the laser status at the start of scan line n is inactive, after

$$m + \frac{T}{2}$$

cycles when the scan line n+1 starts, the laser status becomes active. Therefore, the laser status at the start of each scan line alternates between active and inactive, resulting in interleaved active and dummy pixels.

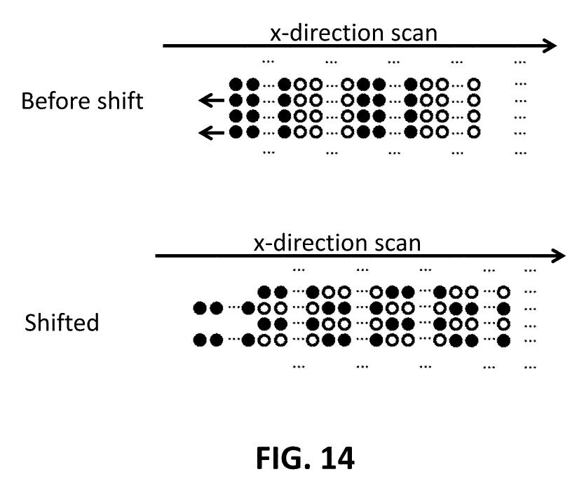

Second, as shown in FIG. 14, a position shift can be introduced for every two (or other predetermined number) x-direction scan lines, where the amount of shift can be set to the number of active pixels. This too results in active and dummy pixels that are interleaved.

At the same time, instead of treating the less than 100% duty cycle as a problem, it may be utilized to reduce the potential phase washout effect while the light beam is scanning across a large range. Theoretically, the smaller duty cycle is less susceptible to phase washout, while it is understood that the smaller duty cycle requires a higher detection bandwidth to accommodate it.

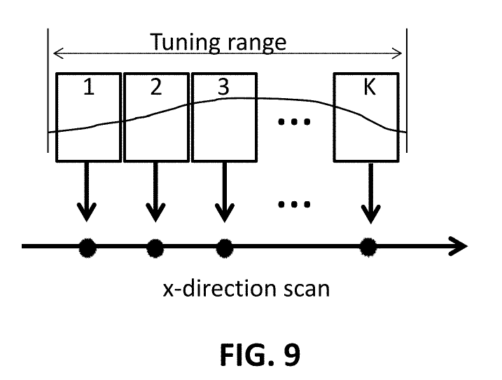

As imaging speed is increased, artifacts caused by eye motion can be decreased. The disclosure herein describes a split spectrum technique to increase the imaging speed by a factor of K, which is a flexible number that can be adjusted according to system specifications and imaging requirements. In order to increase the scanning speed and generation speed of the fundus image, one spectrum of the tunable source 400 is split into K sub-bands, as is illustrated in FIG. 9. The x-direction scan of the 2D scanner 406 can then be synchronized with the sub-bands, by scanning controller 408 in a manner similar to that described above, so that each sub-band generates one pixel in the resulting image. By using this split spectrum method, one full spectrum can generate K pixels. The number of full spectrums required for an M×N image is thus reduced from M×N to M×N/K.

As discussed above, a small wavelength tuning range (e.g., 0.017 nm for 1050 nm light source) is sufficient to differentiate retinal signals from major noises such as cornea reflection. For modern tunable lasers such as swept source lasers with >50 nm tuning range, it is thus possible to split the spectrum into hundreds of sub-bands that are still capable of filtering out reflection noises from the cornea. In practical applications, the number of sub-bands can be flexibly determined depending on several factors including but not limited to: (1) repetition rate of the tunable laser, where a lower repetition rate can be mitigated by increasing the number of sub-bands; (2) total tuning range of the light source, where larger tuning ranges allows larger number of sub-bands; and (3) required imaging speed, where higher imaging speeds can be obtained using a larger number of sub-bands.

It is noted that as the pixel value is statistically calculated, a certain number of data points within each sub-band are beneficial to minimize statistical errors. Computer-based simulation and empirical analysis suggests that this number of data points be greater than or equal to 15.

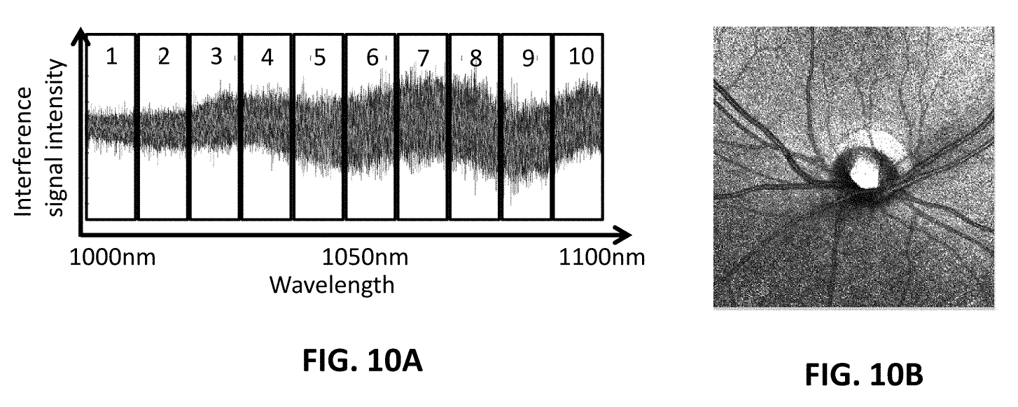

When the whole spectrum is split into K sub-bands, each pixel along the x-direction scan of the 2D scanner 406 corresponds to a sub-band of the full spectrum of the tunable source 400. The value of the pixel can then be calculated by processing a signal of the corresponding sub-band acquired by detector 410. The processing may occur at the detector 410 or by a separate image processor 414. An acquired signal broken down by sub-bands is shown in FIG. 10A. In the example of FIG. 10A, for each full spectrum, the wavelength is tuned from 1000 nm to 1100 nm. The number of sub-bands K is set to 10, which then divides the full spectrum into 10 sub-bands as enclosed by the corresponding boxes. The value of each pixel (each scanning point) is calculated by a statistical measurement, such as the summation of the squared signal intensity within the corresponding sub-band. The resulting en-face image is shown in FIG. 10B. It is noted that the image of FIG. 10B is calibrated as described in more detail below to accommodate the intensity differences between sub-bands.

When using a split spectrum, the light intensity varies for each sub-band. In addition, the actual bandwidth and the number of sampled signal points of each sub-band could also vary for different sub-bands. To generate a better representative image of the fundus, these differences between different sub-bands can be compensated for. This can be addressed by pixel value calibration. One example of a calibration process that may be used after the initial calculation of all the pixel values of the fundus image is described hereinafter. First, pixel values are calculated from the signals within each sub-band. Next, pixels are grouped into K groups according to their sub-bands, and the pixel values for each group are averaged. Finally, pixels are calibrated by dividing the value of each pixel by the corresponding average value for its sub-band.

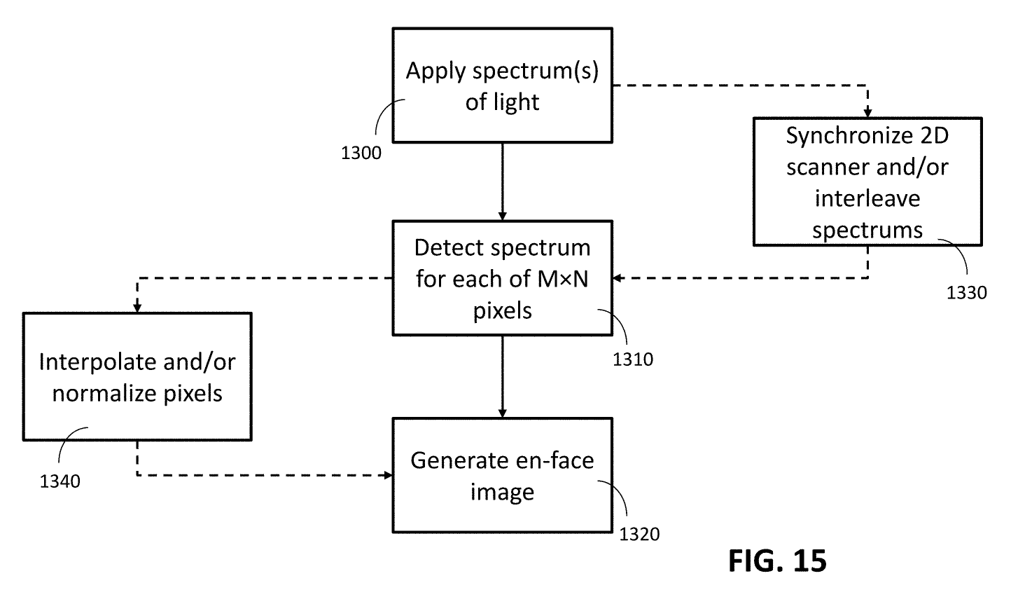

With respect to the above descriptions, it is therefore possible to image an object according to the following method, as illustrated in FIG. 15. The first step 1300 of the method involves applying a spectrum of a light source to an object to be imaged via a two-dimensional scanner. As discussed above, the spectrum of light may be the entire bandwidth of a light source or a sub-band of the light source. When applying the spectrum of light, a two-dimensional scanner may be synchronized with the light source and/or the spectrums of light output by the light source may be interleaved by the two-dimensional scanner 1330, as described above. Next, backscattered light of the spectrum is detected 1310, where the detected light for each applied spectrum corresponds to a pixel of an en-face image having an M×N pixel array. Third, an en-face image is generated 1320, for example by an image processor, from the detected values for each of the pixels. In generating the en-face image, pixels may be interpolated and/or normalized 1340 as described above. The detected values for each of the pixels may be based on the data generated directly by a detector in the imaging system, or otherwise processed data. For example, the processed data may be filtered, have the depth resolution information resolved (e.g., by a Fourier transform), digitized, or any combination thereof. Put another way, the methods described herein are appropriate for generating images from any data, regardless of its level of processing.

The en-face images generated according to the above method may be used for, and just as, en-face images generated by other methods and modalities. According to one example, the en-face images may be used to identify the location of a structure, such as an eye ball, or structures within the eye. If the method is performed iteratively, or otherwise a plurality of en-face images are generated, movement of the identified structure may be tracked by comparing differences between the en-face images. In this manner, the above method can be used, for example, to align and/or track an eye ball.

It should be evident that this disclosure is by way of example and that various changes may be made by adding, modifying or eliminating details without departing from the fair scope of the teaching contained in this disclosure. The invention is therefore not limited to particular details of this disclosure except to the extent that the following claims are necessarily so limited.

What is claimed is:

1. A method of imaging, comprising:
applying a plurality of different spectrums of light from a swept source light source to an object via a two-dimensional scanner;
detecting light of each of the plurality of different spectrums of light that is backscattered by the object, detected light of each applied spectrum of light corresponding to a unique pixel of an en-face image of the object having an M×N pixel array; and
generating the en-face image of the object from data corresponding to the detected light,
wherein the plurality of different spectrums of light each comprise at least one unique wavelength of light.

2. The method of claim 1, further comprising:
synchronizing the two-dimensional scanner with a duty cycle of the light source such that as an output of the light source changes spectrums, the two-dimensional scanner causes the light from the light source to be applied at a different location of the object.

3. The method of claim 1, wherein the two-dimensional scanner does not alter a location of light applied to the object while the light source is inactive.

4. The method of claim 1, wherein an instantaneous linewidth of the swept source light source is smaller than 0.72 nanometers.

5. The method of claim 4, wherein a wavelength tuning range of the swept source light source is larger than 0.017 nanometers.

6. The method of claim 1, wherein each pixel of the en-face image is generated by calculating the sum of the squared signal intensities for the detected light of the spectrum of light corresponding to each pixel.

7. The method of claim 6, wherein the en-face image is generated by normalizing pixels of the M×N pixel array corresponding to each of the at least two different spectrums.

8. The method of claim 1, further comprising:
frequency filtering data corresponding to the detected light to selectively retain a portion of the data corresponding to depths of interest of the object.

9. The method of claim 8, wherein a filtering bandwidth is adjusted based on an estimate of curvature of the object and an evaluation of the en-face image.

10. The method of claim 1, wherein the light is applied and detected according to an interferometric system, the method further comprising:
adjusting a path length of a reference arm of the interferometric system such that the path length of the reference arm and a path length of a detection arm of the interferometric system are equal at varying depths corresponding to a curvature of the object.

11. The method of claim 1, wherein the en-face image is a fundus image.

12. The method of claim 1, wherein the object is an eye ball.

13. The method of claim 1, further comprising aligning and/or tracking an eye ball based on the generated en-face image, wherein the method is performed at least in part by an interferometric system.

14. The method of claim 1, further comprising digitizing each detected spectrum at at least 15 sample points within the spectrum, the en-face image being generated at least in part from the digitized sample points.

15. A method of imaging, comprising:
detecting spectrums of light that are backscattered by an object at various depths of the object, each detected spectrum of light corresponding to a unique pixel of an en-face image of the object having an M×N pixel array and being output by a swept source light source;
filtering data corresponding to the detected spectrums of light by applying a frequency filter corresponding to a depth of interest;
selectively retaining the filtered data; and
generating the en-face image of the object by performing a statistical calculation on the selectively retained data.

16. The method of claim 15, wherein the spectrums of light are the same.

17. The method of claim 15, wherein the spectrums of light comprise at least two different spectrums within the bandwidth of the swept source light source, the at least two different spectrums each comprising at least one unique wavelength of light.

18. The method of claim 15, further comprising:
synchronizing the two-dimensional scanner with a duty cycle of the light source such that as an output of the light source changes spectrums, the two-dimensional scanner causes the light from the light source to be applied at a different location of the object.

19. The method of claim 15, wherein the two-dimensional scanner does not alter a location of light applied to the object while the light source is inactive.

20. The method of claim 15, wherein an instantaneous linewidth of the swept source light source is greater than 0.72 nanometers.

21. The method of claim 15, wherein a wavelength tuning range of the swept source light source is less than 0.017 nanometers.

22. The method of claim 15, wherein each pixel of the en-face image is generated by calculating the sum of the squared signal intensities for the detected light of the spectrum of light corresponding to each pixel.

23. The method of claim 22, wherein the en-face image is generated by normalizing pixels of the M×N pixel array corresponding to each of the at least two different spectrums.

24. The method of claim 15, wherein a bandwidth of the frequency filter is adjusted based on an estimate of curvature of the object and an evaluation of the en-face image.

25. The method of claim 15, wherein the light is applied and detected according to an interferometric system, the method further comprising:
adjusting a path length of a reference arm of the interferometric system such that the path length of the reference arm and a path length of a detection arm of the interferometric system are equal at varying depths corresponding to a curvature of the object.

26. The method of claim 15, wherein the en-face image is a fundus image.

27. The method of claim 15, wherein the object is an eye ball.

28. The method of claim 15, further comprising aligning and/or tracking an eye ball based on the generated en-face image, wherein the method is performed at least in part by an interferometric system.

29. The method of claim 15, further comprising digitizing each detected spectrum at at least 15 sample points within the spectrum, the en-face image being generated at least in part from the digitized sample points.
